(19) United States
(12) Patent Application Publication
(10) Pub. No.: US 2004/0033252 A1
(43) Pub. Date: Feb. 19, 2004

(54) Agents for recoverying from or preventing fatigue in the central nerve system and foods for recovering from or preventing fatigue

(76) Inventors: Takanobu Yamamoto, Nara (JP); Eric A. Newsholme, Oxfordshire (GB)

Correspondence Address: WENDEROTH, LIND & PONACK, L.L.P., 2033 K STREET N. W., SUITE 800, WASHINGTON, DC, 20006-1021, US

(21) Appl. No.: 10/415,286

(22) PCT Filed: Oct. 26, 2001
(86) PCT No.: PCT/JP01/09439

(30) Foreign Application Priority Data
Oct. 27, 2000 (JP) 2000-329409
Apr. 17, 2001 (JP) 2001-118710

(51) Int. Cl. A61K 47/00; A61K 31/195

(52) U.S. Cl. 424/439; 514/561

(57) ABSTRACT

The present invention provides a fatigue-preventive (brain fatigue) agent or a fatigue-recovering agent for the central nervous system, which contains a branched-chain amino acid, such as L-valine, L-leucine and L-isoleucine, and/or a 2-aminobicyclo-[2,2,1]-heptane-2-carboxylic acid. In addition to the application as injection and transfusion, these can be prepared as solid-state modes to be taken, such as tablets, pellets and powder medicine by adding an appropriate vehicle such as starch and lactose thereto. Moreover, these can be prepared as various beverages such as so-called health drinks and sports drinks, or as food items for fatigue-recovering and the fatigue-prevention in the central nervous system.

### TECHNICAL FIELD

The present invention relates to a fatigue-recovering or fatigue-preventive agent for a central nervous system, a food for recovering from fatigue of the central nervous system, a food for the fatigue prevention for the central nervous system and a screening method for an inhibitory substance for fatigue of the central nervous system, and also concerns use of the rat as a fatigue model for the central nervous system.

### TECHNICAL BACKGROUND

Conventionally, in an attempt to muscles assist from the fatigue, various dietary supplements in which, for example, various metal ions such as potassium ions, and sodium ions, sugar, amino acids and the like are blended, have been developed.

However, these dietary supplements are intended for recovery from physical (muscular) fatigue, and are not intended to directly recovery from fatigue in the central nervous system.

In recent years, the fatigue in the central nervous system, such as chronic fatigue syndrome (CFS), information fatigue syndrome, information stress syndrome and Internet dependency, have received a great deal of attention. In these cases, the fatigue in the central nervous system results from the fatigue occurred in a large portion of intercerebral control circuits caused by suppression in a level of voluntary exciting, which are suppressed in the number of motor units to the level of voluntary neuromuscular junction-muscle fibers and the firing frequency, that is, a fatigue different from the fatigue in the motile muscles themselves. Moreover, this fatigue is different from so-called the tiredness feeling caused by physical (muscular) fatigue, and is generated in a state that is not accompanied by physical fatigue, such as computer work or reading. The mechanism of this fatigue in the central nervous system has not been sufficiently clarified.

The inventors of the present invention have clarified the mechanism of this fatigue in the central nervous system, found that a branched-chain amino acid and 2-aminobicyclo[2,2,1]heptane-2-carboxylic acid that is a specific inhibitor of the L-system transporter on BBB make it possible to suppress the fatigue in the central nervous system, and also proved that, in particular, the application of both of these in combination makes it possible to virtually suppress the fatigue completely; thus, the present invention has been devised. It has been proved that the pharmacological effects are based upon the synergism (potentiation) of the two components.

Moreover, during its testing processes, it has been found that analbuminemia rats that have no albumin potentially and tryptophan-deficient rats are useful as a fatigue-model with respect to the central nervous system, and that treadmill running tests using these rats can be utilized as a screening method for an inhibitory substance against fatigue in the central nervous system.

### DISCLOSURE OF THE INVENTION

An agent for recovering from fatigue of the central nervous system and a fatigue preventive agent for the central nervous system, relating to the present invention, are characterized by containing a branched-chain amino acid and/or 2-aminobicyclo-[2,2,1]-heptane-2-carboxylic acid.

Moreover, a food for recovering form fatigue of the central nervous system and a food for preventing fatigue in the central nervous system, relating to the present invention, are characterized by containing a branched-chain amino acid and/or 2-aminobicyclo-[2,2,1]-heptane-2-carboxylic acid.

An inhibitory substance based screening method for fatigue in the central nervous system relating to the present invention is characterized by measuring the fatigue-inhibiting degree by the treadmill running tests using analbuminemia rats or tryptophan-deficient rats.

Moreover, the fatigue-model rats with respect to the central nervous system in accordance with the present invention are characterized by preparing analbuminemia rats or tryptophan-deficient rats.

### BRIEF DESCRIPTION OF THE DRAWINGS

FIG. 1 is a drawing that shows the relationship between sites of action of BCH and BCAA and the synergism thereof.

### BEST MODE FOR CARRYING OUT THE INVENTION

An agent for recovering from fatigue of the central nervous system and a fatigue preventive agent for the central nervous system, relating to the present invention, are characterized by containing a branched-chain amino acid and/or 2-aminobicyclo-[2,2,1]-heptane-2-carboxylic acid (hereinafter, referred to as “BCH” in the present invention).

Moreover, a food for recovering from fatigue of the central nervous system and a food for preventing fatigue in the central nervous system, relating to the present invention, are characterized by containing a branched-chain amino acid and/or 2-aminobicyclo-[2,2,1]-heptane-2-carboxylic acid.

In the present invention, the fatigue in the central nervous system has been defined as described above, and the fatigue preventive agent (fatigue preventing food) refers to an agent that is mainly applied to a human being before a state in which the fatigue in the central nervous system is anticipated, while the recovering agent (recovering food) refers to an agent that is mainly applied to a human being when the fatigue in the central nervous system has occurred. In other words, the fatigue recovering agent and the fatigue preventive agent for the central nervous system of the present invention can be applied with or without fatigue, and used not only in the medical field, but also in the food field including so-called sports drinks. In particular, the agents are expected to be used in new applications, that is, as specific health and physical food so as to prevent and recover from the fatigue in the central nervous system (brain fatigue).

In the present invention, with respect to the branched-chain amino acid (BCAA), essential amino acids for a human body, such as L-valine, L-leucine and L-isoleucine, which have a branched chain in its carbon chain, are used. Moreover, with respect to these amino acids, salts thereof that are physiologically permissible, for example, hydrochloric acid salts and various hydrates thereof, can also be used. These branched-chain amino acids can be respectively used alone, or in combination as a mixture, and, preferably used as a mixture of three components, that is, L-valine, L-leucine and L-isoleucine. In the case of the application as a mixture, the ratio of the mixture of these is not particularly limited. With respect to the dose of administration of the branched-chain amino acids, in the case of a human being, a BCAA is set to approximately 10 to 1,000 mg/kg,more preferably, approximately 50 to 500 mg/kg.

Moreover, for example, as suggested by reports written by Kanai and Endo (Japanese Journal of Pharmacol., Vol. 82, Supplement I, 8p, 2000), the BCH is applicable as an antitumor agent (in the same manner as the inventors of the present invention, applied so as to suppress an L-system transporter in vivo, that is, to suppress an amino acid transporting system to tumor cells), and this substance is considered to be applicable to the human body safely. In particular, the amount of application of the BCH is as small as approximately 1/10 to 1/100 of the amount of application of branched-chain amino acid; therefore, this substance is very effective.

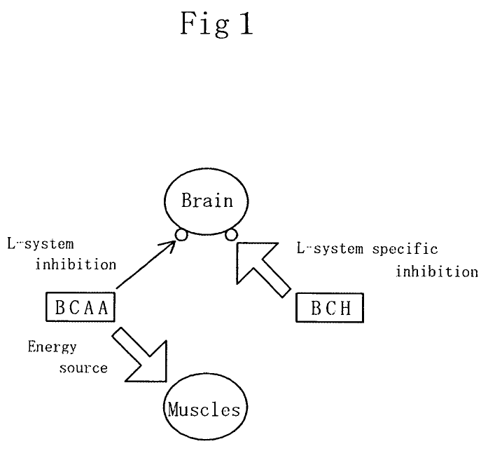

These branched-chain amino acid and the BCH can be solely used as a fatigue recovering agent and a fatigue preventive agent for the central nervous system or food used for the purpose of the fatigue recovery and the fatigue prevention; however, the application of both of these in combination makes it possible to positively exert functions as a fatigue recovering agent and a fatigue preventive agent for the central nervous system or food used for the purpose of the fatigue recovery and the fatigue prevention more effectively (synergistic effects, see FIG. 1).

With respect to application and administration modes of the fatigue recovering agent and the fatigue preventive agent of the present invention, not particularly limited, any modes can be used as long as they are applicable to the human being. These can be prepared as, for example, injection or transfusion that are directly administered to the blood circulatory system and the lymphoid system, or an appropriate excipient such as starch and lactose can be added thereto so as to form solid-state modes to be taken, such as tablets, pellets and powder medicine. Moreover, these can be prepared as various beverages such as so-called health drinks and sports drinks, or as the fatigue recovering and the fatigue preventing food items prepared as food items, such as biscuits, candies and jellies.

Moreover, to the fatigue recovering agents and the fatigue preventive agents and food items for the fatigue recovery and the fatigue prevention of the present invention, various compounds that have been conventionally used for physical recovering, such as various amino acids other than branched-chain amino acids and BCH, sugars such as glucose and saccharose, various vitamins such as vitamin B1, vitamin B2 and vitamin C, and metal ions and the like, such as sodium ions, potassium ions and calcium ions, can of course be added.

Next, the inhibitory substance screening method of the fatigue in the central nervous system in the present invention is characterized by measuring the fatigue-inhibiting degree by treadmill running tests using analbuminemia rats or tryptophan-deficient rats. Here, the analbuminemia rats lack albumin in their blood plasma (intrinsic factor), and, for example, include those rats suffering from genetic albumin-deficient in their blood plasma. The preparation method for such rats has been known (Nagase et al.; J. Biochem. 94, 623-632, 1983, et al.), and those rats are available in the market prepared by, for example, Japan SLC Inc. Moreover, those tryptophan-deficient rats can be obtained by the following method: rats which had been fed with the tryptophan-containing normal food for a predetermined period after birth were grown to a weight of approximately 170 to 230 g, more preferably, 200 g±10 g (normally, one-month old after birth), and were then switched to tryptophan-lacking food, and kept without feeding tryptophan for at least two weeks. Thus, these rats were raised without tryptophan. For example, as will be described in example 2, these rats were raised for one month after birth to have a weight of 200 g, and then fed with the tryptophan-lacking food for at least two weeks; thus, target rats were obtained. These rats were fed with the minimum tryptophan that was considered essential for growth. In comparison with those rats having been fed with the tryptophan-containing food, these rats had a low concentration of tryptophan in their extra-cellular fluid so that it was possible to suppress influences due to endogenous tryptophan.

These rats were subjected to treadmill running tests so that the fatigue degree (time before fatigue was reached) was measured; thus, it was possible to measure the degree of the fatigue in the central nervous system. The intracerebral transfer process of the tryptophan is expected to be intervened by albumin, and as will be described later, the tryptophan is considered to form a fatigue substance in the central nervous system. For this reason, in an attempt to measure the central nervous system fatigue inhibition of a target substance, in the case of normal rats having endogenous tryptophan, it is not possible to eliminate influences of albumin, resulting in failure when accurately measuring central nervous system fatigue inhibition. In contrast, when the central nervous system fatigue inhibition of a target substance is measured using rats that have been subjected to tryptophan deficiency, it was supposed that originally, there was no difference between a target-substance applied group and a no-application group. However, in an attempt to measure the combined function between the target substance and the BCAA, it has been found that a drastic fatigue inhibition is generated due to the combined function. This means that by comparing the evaluation of the BCAA single application (the fatigue inhibitory effect=increase in time up to the fatigue state) with the evaluation in the combined application of the BCAA and the target substance, it is possible to search for a specific fatigue inhibitory substance to the central nervous system. Of course, with respect to models relating to the fatigue in the central nervous system by the use of tryptophan, not limited to the combined effects with the BCAA, various applications are proposed, including measurements on the fatigue inhibitory effects by the use of a single substance.

Thus, by measuring the degree of the fatigue inhibitor by treadmill running tests by using the analbuminemia rat or tryptophan-deficient rats, it is possible to accurately measure the fatigue inhibitory effects with respect to the central nervous system, and also to apply this as a screening method of the fatigue inhibitory substance.

### EXAMPLES

Next, the following experiments were carried out so as to confirm the effects of the present invention.

### Experimental Example 1

Three-week-old female analbuminemia rats (Japan SLC) that genetically lacked albumin in their blood plasma, which had been raised under light and dark cycles of 7:00 to 19:00 (light cycle) at room temperature 22° C., were subjected to the fatigue tests. Prior to the fatigue tests, the analbuminemia rats were subjected to training (20 m/min, 7% inclination) for 30 minutes, four times a week, during a predetermined period of time in 13:00 to 15:00, and this training lasted for four weeks so as to adapt them to running on the treadmill. After the training had been completed, all the analbuminemia rats (weight 210 to 255 g) were subjected to exhaustion tests on the treadmill under the same conditions, and time up to an exhaustion state was measured. The exhaustion state was defined as the point of time at which the rat failed to follow the speed of the treadmill or the point of time at which the rat refused to run.

The rats, which were divided into four groups, were respectively treated with physiological saline (made by Otsuka Pharmaceutical Co., Ltd “0.9% physiological saline”), the BCH (made by Sigma K.K., NoA7902), the BCAA and albumin (made by Sigma K.K. A-6272 (Fraction V)). The dose of physiological saline was set to 5 ml/kg, that of the BCH was 8 mg/kg and that of the BCAA was 250 mg/kg, and these were given into the abdominal cavity one hour prior to the start of the running tests. Here, BCH, BCAA and albumin were respectively dissolved in physiological saline, and then administered. The dose of albumin was set to 1 g/kg, and given into the abdominal cavity one and half hours prior to the start of training. Moreover, the BCAA was used as a mixture of L-valine, L-leucine and L-isoleucine (weight ratio, 5:3:2, respectively special class reagents made by Wako Pure Chemicals Industries, Ltd.). These doses were determined so as to obtain sufficient effects, and in the case of actual doses to the human being, these can be changed appropriately depending on various factors.

The head of each of the rats was sacrificed by decapitation immediately after the training so that the striatal synaptosome was separated, and tryptophan(Trp), 5-hydroxytryptophan (5-HTP), 5-hydroxytryptamin (5-HT) and 5-hydroxyindoleacetic acid (5-HIAA) were measured by high-performance liquid chromatography using an electrochemical detector. Moreover, the protein level in the P2 fraction was measured in accordance with the method of Lowry et al. (J. Protein measurements with the Folin phenol reagent, J. Biol. Chem. 193: 265-275; 1951).

These measured data were obtained by a analysis of variance (ANOVA) in one-way layout of Fisher's PLSD test by using multiple comparisons and repeated measurements, based upon the standard error. Next, the data were classified based upon running times, and analyzed by the Student's t-test based upon observation with paired t-test in the total effects of the BCAA and BCH treatment groups. Tables 1 and 2 show the results of the analyses.

TABLE 1
Running time up to exhaustion in NAR's that were treated with
physiological saline, albumin, BCAA and BCH

| | Running time up | | |
|---|---|---|---|
| | to exhaustion | | |
| | (min.) | Range (min.) | P |
| Physiological saline (n = 6) | 93 ± 17 | 40-139 | |
| Albumin (n = 6) | 96 ± 22 | 41-182 | ns |
| BCAA (n = 5) | 188 ± 24 | 115-251 | <0.05* |
| BCH (n = 5) | 188 ± 33 | 94-271 | <0.05* |

NAR; Nagase genetically analbuminemia rats
BCAA; Branched-chain amino acid
BCH; 2-aminobicyclo [2,2,1] heptane-2-carboxylic acid
ns; no significant difference
Data represent a difference from the physiological saline level. Then, the value is represented as mean values ± SEM. The analysis of variance in one-way layout was carried out by Fisher's PLSD test.
*F(3, 18) = 3.21

TABLE 2
Concentrations of the tryptophan and its metabolites in striatal
synaptosome immediately after exhaustion in NAR's that are
treated with physiological saline, albumin, BCAA and BCH

| | tryptophan | 5-HTP | 5-HT | 5-HIAA |
|---|---|---|---|---|
| Physiological saline (n = 6) | 56.27 ± 3.54 | 1.12 ± 0.05 | 4.52 ± 0.87 | 2.90 ± 0.25 |
| Albumin (n = 6) | 52.41 ± 8.97 | 1.45 ± 0.18 | 5.00 ± 0.47 | 3.30 ± 0.06 |
| BCAA (n = 5) | 43.70 ± 2.07 | *0.79 ± 0.06* | 5.14 ± 1.27 | 3.32 ± 0.25 |
| BCH (n = 5) | 53.43 ± 10.50 | 1.25 ± 0.17 | 5.20 ± 0.79 | 3.58 ± 0.21 |

NAR; Nagase genetically analbuminemia rats
BCAA; Branched-chain amino acid
BCH; 2-aminobicyclo [2,2,1] heptane-2-carboxylic acid
5-HTP; 5-hydroxytryptophan
5-HT; 5-hydroxytryptamine
5-HIAA; 5-hydroxyindoleacetic acid
ns; no significant difference
Data represent a difference from the physiological saline level. Then, the value is represented as mean values (pmol/mg protein) ± SEM. ANOVA in one-way layout was carried out by Fisher's PLSD test.
*p < 0.05; +p < 0.05

As shown in Table 1, in both of the BCAA treated group and the BCH treated group, a significant increase was observed in the running time to exhaustion. Moreover, in the BCAA treated group, the time up to exhaustion was significantly prolonged in comparison with the physiological saline treated group, the concentrations of the tryptophan and 5-HTP in the striatal synaptosome in the BCAA treated group are significantly reduced with respect to the physiological saline treated group [−22%: F(3, 18)=2.08, p<0.05 and −29%: F(2, 13)=7.08, p<0.05]. Moreover, there was no significant difference between the albumin treated group and the BCH treated group; however, there was a great variation in the standard error in the tryptophan concentration.

Next, the measured data were analyzed by classifying times up to exhaustion (group A: 40 to 189 minutes, B: 190 to 271 minutes). In this case, in comparison with the physiological saline treated group, upon totaling the BCAA treated group and the BCH treated group, there was a significant reduction in the uptake of the tryptophan (−19%: d.f.=9, p<0.05) and 5-HTP (−23%: d.f.=9, p<0.025) into the striatal synaptosome ingroup B. Moreover, in the rat having the shortest running time in the group (the above-mentioned A group) treated with the BCAA or the BCH, there was no difference in the concentrations in synaptosome of the tryptophan and 5-HTP. In other words, there was a significant variation in the uptake level of the tryptophan in the striatal synaptosome between the analbuminemia rat having the longest duration and the analbuminemia rat having the shortest running time. The above-mentioned experiments show that the BCAA and the BCH are expected to suppress the uptake of tryptophan, alleviate fatigue in the central nervous system and improve the endurance capacity.

It has been known that the fatigue in the central nervous system shows not a reduction in the serotonergic-system function in the central and peripheral nerves, but, in contrast, an enhanced nerve transmission response, and that this implies a relation to a change in the transmission of extracellular fluid 5-HT that depends on an increase in the tryptophan. This change in the transmission of extracellular fluid 5-HT causes suppression in the surrounding brain nerves, resulting in fatigue in the central nervous system (“Tryptophan/5-HT Hypothesis”).

Here, with respect to the control of the tryptophan transport into the brain in peripheral nerves, the following factors are taken into consideration: (a) a change in the binding affinity of the tryptophan to albumin and (b) competition between the BCAA and the tryptophan in the blood plasma that is transmitted through the L-system transporter with respect to entrance into the brain. Therefore, it is considered that an increase in the albumin concentration exerted by the exogenous albumin administration with using analbuminemia rat and/or an increase in the BCAA concentration exerted by the exogenous BCAA administration make it possible to control the uptake and transport of the tryptophan into the brain.

In this manner, the fatigue in the central nervous system might be caused by a reduction in the albumin level and the above-mentioned bonding affinity. Here, the fatigue in the central nervous system might be diminished if the albumin concentration in blood increases; however, the above-mentioned experiments show that the fatigue in the central nervous system was not improved in the analbuminemia rats through the administration of albumin. Moreover, in comparison with the BCAA and the BCH treatments, the albumin treatment did not inhibit the uptake of the tryptophan into synaptosome, and no advantageous effects to the fatigue were observed in the analbuminemia rats.

Moreover, in the same manner as the BCAA treatment, the BCH treatment provided a very long running time, and resulted in a reduction in the concentration of the tryptophan and 5-HTP in synaptosome. The BCH serves as a specific inhibitor against an L-system transporter that is one of the amino-acid transporting systems or an analog form of leucine, and these two factors are considered to be obtained from not peripheral effects as an energy source, but from the inhibition of the L-system transporter on BBB.

As described above, as shown in Table 2, the administration of the BCAA and the BCH provided reductions in the uptake of the tryptophan and the synthesis of 5-HT that relate to the fatigue in the central nervous system by 19% and 23% respectively, and as shown in Table 1, helped to extend the running time to approximately twice as long. In the analbuminemia rats, the tryptophan concentration and the tryptophan dynamics in blood plasma were not affected by albumin so that it became possible to eliminate effects of the endogenous albumin control.

Here, in the present method using treadmill running tests, it is considered that the fatigues in both of the central system (central nervous system) and the peripheral system (muscle system) exist in a related manner. The tryptophan, which is a causal substance of the fatigue, is transferred from the peripheral system (in blood) to the central system (brain) through the blood brain barrier (L-system transporter) to give inhibiting (negative) information to the central nervous system. As a result, actions are suppressed; that is, the fatigue phenomena, derived from the central system, appears. In other words, an excessive amount of the tryptophan or 5-HT in the brain suppresses the central nervous system, causes a reduction in the motor system output that is released through pyramidal tracts and x-motor neurons, and finally inhibits the treadmill running performance, that is, causes the fatigue phenomena derived from the central nervous system. In this manner, it is considered that the present method is appropriate in observing fatigue in the central nervous system. However, since muscle organisms are partially involved, the fatigue characteristic is a psychosomatic monistical characteristic including peripheral nerves. Moreover, since the tryptophan signal from the peripheral nerves to the brain is inhibited (controlled) on the L-system transporter by using the BCH and the BCAA, the above-mentioned experiments are clearly related to the central fatigue. Thus, the above-mentioned experiments have substantiated that the BCAA and the BCH appropriately contribute to recovering from fatigue of the central nervous system by eliminating influences from exogenous and endogenous albumin and influences of the uptake of the tryptophan into the brain; consequently, these can be used solely, or can be used in combination so as to prevent the fatigue in the central nervous system and recover the fatigue in that.

Moreover, the application of analbuminemia rats makes it possible to eliminate influences from endogenous albumin, and it has been confirmed that a method in which running time of the analbuminemia rats is measured in treadmill running tests is utilized as a fatigue model in the central nervous system.

### Experimental Example 2

Three-week-old female rats of Sprague-Dawley-type (each rat: 50 g) were raised for one month to a weight of 200 g [fed with normal food of AIN93G for one month (standard refined feed, made by Oriental Yeast, Co., Ltd., with the tryptophan being contained at 2.3 g/kg)], and from this time on, these were fed with the tryptophan-deficient food (adjusted feed in which only the tryptophan was removed from the above-mentioned AIN93G, with corn starch being used for compensating for the corresponding portion) for 16 days to form the tryptophan-deficient rats. Amino acids, contained in the normal food AIN93G, were respectively manufactured by AJINOMOTO CO., INC., and the respective contents (g) in 1 kg of feed were: alanine 5.6, arginine 6.8, aspartic acid 13.1, cystine 3.9, glutamic acid 39.6, glycine 3.4, histidine 5.6, isoleucine 10.1, leucine 17.5, lysine 14.9, methionine 5.6, phenyl alanine 9.5, proline 21.6, serine 9.7, threonine 7.7, tryptophan 2.3 (only contained in the tryptophan-containing food), tyrosine 10.4, and valine 12.6 (each of the amino acids having a purity of 100%). These rats were subjected to training for 30 minutes at a speed of 20 m/min (inclination: 7%) by treadmill running practice, three times a week, which started at the age of 3 weeks old, and lasted for 2 months. After the training had been completed, the rats were subjected to running loads on the treadmill at a speed of 20 m/min (inclination: 7%) up to exhaustion, and the running time was evaluated as the fatigue tests shown below. The exhaustion state was defined as the point of time at which the rat failed to follow the speed of the treadmill or the point of time at which the rat refused to run. The evaluation was made by using inter-group comparisons (with paired t-test) of a Student's t-test.

(Evaluation Test 1)

In order to confirm influences of the ingested tryptophan, comparisons were made between control rats which were raised by feeding on normal food from the point of time at which they had been raised to a weight of 200 g and subjected to the above-mentioned training and the tryptophan-deficient rats. Table 3 shows the results thereof. Here, the concentration of the tryptophan and the concentration of 5-HIAA (metabolites of the tryptophan and serotonin) in striatal extracellular fluid of the tryptophan-deficient rats that had been raised with the tryptophan-lacking food were reduced to respectively 55% and 53% of the corresponding concentrations, when compared with rats that had been raised with the tryptophan-containing food.

TABLE 3
Running time up to exhaustion between rats raised with the
tryptophan-deficient food and rats raised with normal food

| | Running time up | |
|---|---|---|
| | to exhaustion (min.) | P |
| tryptophan-deficient food group (n = 5) | 323 ± 31 | p < 0.025 |
| Normal food group (n = 4) | 224 ± 10 | |

Student's t-test; t = 3.039; d.f. = 7
Data is represented as mean values ± standard error

(Evaluation Test 2)

To the tryptophan-deficient rats that had been subjected to the above-mentioned training were respectively given physiological saline, a mixture of the BCH and BCAA, the BCH and the BCAA, and comparison tests were carried out among the four groups. The dose of physiological saline was set to 5 ml/kg, that of the mixture of the BCH and BCAA was set to 240 mg/kg, that of the BCH solely used was set to 150 mg/kg, and that of the BCAA solely used was set to 250 mg/kg, and these were given into the abdominal cavity one hour prior to than the start of the running tests. Here, the BCH, the BCAA and the albumin were respectively dissolved in physiological saline, and then applied. Moreover, the BCAA was used as a mixture of L-valine, L-leucine and L-isoleucine (weight ratio, 5:3:2), and the BCH and the BCAA were mixed so that the BCH was 37.5% by weight with the BCAA being 62.5% by weight. Table 4 shows the results thereof.

TABLE 4
Running time up to exhaustion in the tryptophan-deficient rats that
are treated with physiological saline, BCAA and BCH

| | Running time up | |
|---|---|---|
| | to exhaustion (min.) | P |
| Physiological saline (n = 5) | 323 ± 31 | |
| BCH (n = 5) | 308 ± 22 | ns |
| BCAA (n = 5) | 322 ± 18 | ns |
| Mixture of BCH and BCAA (n = 5) | >480 (n = 4), 402 (n = 1) | |

BCH; 2-aminobicyclo [2,2,1] heptane-2-carboxylic acid
BCAA; Branched-chain amino acid
ns; no significant difference
Data represent a difference from the physiological saline level. Then, the value is represented as mean values ± standard error. Inter-group difference was obtained by a Student's t-test.

(Evaluation Test 3)

To the tryptophan-deficient rats that had been subjected to the above-mentioned training were respectively given tryptophan-added food (AIN93G to which 2.3 g/kg of the tryptophan was added), and were then given physiological saline, a mixture of the BCH and the BCAA, the BCH and the BCAA, and comparison tests were carried out among the four groups. The dose of each of these was set in the same manner as the above-mentioned evaluation test 2, and these were given into the abdominal cavity one hour prior to than the start of the running tests. Table 5 shows the results thereof.

TABLE 5
Influences of tryptophan-containing food on running time up to
exhaustion in the tryptophan-deficient rats that have been
treated with physiological saline, BCAA and BCH

| | Running time up | |
|---|---|---|
| | to exhaustion (min.) | Range (min.) |
| Physiological saline (n = 4) | 387 ± 25 | 340-443 |
| Mixture of ECH and BCAA (n = 5) | >542* | >542* |
| BCH (n = 5) | 428 ± 45*** | 306->542** |
| BCAA (n = 5) | 447 ± 4**** | 430-452 |

BCH; 2-aminobicyclo [2,2,1] heptane-2-carboxylic acid
BCAA; Branched-chain amino acid
The value is represented as mean values ± standard error.
*All the rats have not reached exhaustion even after the running time of 542 minutes.
**In one example of BCH solely-administered groups, the rats have not reached exhaustion even after the running time of 542 minutes.
***No significant difference from physiological saline administered groups.
Data were obtained through Student's t-tests. Here, the mean values was obtained by setting values > 542 as 542.
****p < 0.05, t = 2.369, d.f. = 7

(Results of Evaluation)

In experimental example 1, it has been confirmed that, when the administration of the BCH and the BCAA inhibits the transport of the tryptophan from blood into the brain through the competitive inhibition against L-system transporter that is a transporter of neutral amino acids located on the blood brain barrier (BBB), it is possible to suppress an increase in the tryptophan signal serving as a central fatigue substance, and to contribute to recovery from fatigue or fatigue prevention for the central nervous system. In the above-mentioned experimental example 1, the BCH treatment and the BCAA treatment are carried out on the premise of “Tryptophan/5-HT Hypothesis” with respect to the fatigue in the central nervous system; therefore, it is considered that the effects of these treatments are not expected in the rats that have been raised by giving tryptophan-deficient food. Table 3 and Table 4 substantiate these conclusions, and as shown in Table 3, the tryptophan-deficient rats had a longer period of time to reach exhaustion in comparison with rats fed with normal food, thereby substantiating the influences of the tryptophan to exhaustion. Moreover, as clearly shown by Table 4, in the tryptophan-deficient rats, no significant difference exists between the physiological saline administered group (control group) and the BCH or the BCAA administered groups.

However, only in the mixed BCH and BCAA administered group, 4 examples were observed in which even after a lapse of 8 hours (480 minutes), the rats had not reached exhaustion (Table 4). Probably, even in the case of the tryptophan-deficient rats, a slight amount of pooled tryptophan, made during the initial stage of the growing period in which normal food has been given thereto, has a great influence on the fatigue in the central nervous system; therefore, it is considered that these synergistic functions from the combined administration have made rats that are not susceptible to the fatigue. In the case of the BCAA only-administration, BCAA also contributes as an energy source for muscles, since there is no difference from the physiological saline administered group, the effects of the BCH on the synergistic functions exerted by the combined administration, are considered to be completely related to the central nervous system.

Next, in the case when the tryptophan-containing food is given to the tryptophan-deficient rats, as shown in Table 5, the BCAA only-administration significantly exerts the extension effects up to exhaustion statistically (p<0.05, t=2.369, d. f.=7). Moreover, the case of the BCH solely-administration causes a running-time difference of approximately 40 minutes in the average-value comparison between the two groups, although no statistically significant difference is observed. Furthermore, two cases (524 minutes and not less than 524 minutes) in which rats have not reached exhaustion caused only by the BCH administration were observed. In contrast, easily-fatigued rats were observed so that there was a great individual difference in the BCH effects. This is due to characteristics in the fatigue tests by the treadmill running practices used in the present experiments. In other words, as described earlier, the present tests are considered to include two factors, that is, “the fatigue of the muscles themselves” and “the fatigue in output information from the central nervous system to the muscles”, and these facts are caused by the existence of the two factors in a combined manner. Therefore, it is considered that, when the fatigue in the central nervous system is blocked during treadmill running, some rats have an extended period up to exhaustion while other rats fail to continue the treadmill running due to the fatigue of muscles themselves prior to the fatigue in the central nervous system. In order to compensate for this defect, the specific alleviating function on the fatigue in the central nervous system is strengthened by using the BCH, and this effect is further reinforced by the BCAA; thus, as shown in Table 5, it becomes possible to form rats that are almost free from the fatigue. Probably, the specific inhibition against the L-system transporter by the BCH causes a reduction in the input to the central tryptophan dependent “the fatigue signal transduction”, with the result that the motor-system output information (nerve signal to voluntary muscles) from the intracerebral control circuit is continuously sent to the lower level (α-motor neuron that is a final common path). Consequently, it is concluded that “no fatigue occurs”. In this manner, in an attempt to confirm the effects to the tryptophan that forms a target of the BCH and the BCAA administration, the formation of the tryptophan-deficient rats is very useful.

Conventionally, the BCAA has been applied to pathologic fluid therapy as venous nutrient agents, and it has been known that in addition to the effect that its keto acid is utilized as an energy base in the skeletal muscles at the time of a damage to muscles, the BCAA also contributes to synthesis of other amino acids and proteins as a supply source of nitrogen. Therefore, the BCAA not only contributes to prevention of muscle fatigue and recovering, but also provides sites of action on the L-system transporter (as described in experimental example 1, published in Brain Research Bulletin, 52(1), 35-38, 2000, by the inventors, of the present application) so that the BCAA is effective on both the brain and muscles; moreover, the present experiments have proved that, by strengthening and compensating for the specific inhibiting effect on the L-system transporter on BBB using BCH, the two substances make it possible to exert not simple added effects, but “synergistic (potentiation) effects”, thereby providing an effective fatigue-preventive-recovering agent.

As described above, the administration of the BCH and BCAA in a combined manner makes it possible to exert superior effects that would not be achieved by the BCH only-administration and the BCAA only-administration, and this method is based upon a completely novel idea without the necessity of taking into consideration ratio distribution and quantitative blends of respective amino acids that have been required in the BCAA only-administration; therefore, this method is applicable not only to the medical field, but also to various food items, in particular, specific health and physical food items relating to completely new field, that is, the fatigue-prevention and the fatigue-recovering in the central nervous system.

FIG. 1 shows the mechanism of these synergistic functions. The thickness of an arrow in FIG. 1 shows the strength of the effects, and in the case when two kinds of medicines derived from a mixture of the BCH and the BCAA having similar effects on BBB are exerted, the effects are exerted as a sum (added function) of the respective independent functions or as a value greater than the sum (synergistic functions). It is considered that the function of mixed BCH and BCAA makes it possible to synergistically act on the central fatigue on the L-system transporter to alleviate the fatigue. As shown in Table 5, the effects of a combined administration of the BCH and the BCAA are substantiated by all the rats in the 5 examples used in the experiments, which are confirmed to be still free from the fatigue even after the treadmill running practices of not less than 9 hours (542 minutes); thus, it is confirmed that the mixed administration of the BCH and the BCAA makes it possible to effectively contribute to the fatigue-prevention and fatigue-recovering in the central nervous system.

An excessive amount of intracerebral tryptophan enhances the synthesis of 5-HT, and the change in its transmission might induce suppression in surrounding brain nerves, and the possibility of this function has been explained in some portions in the above description; moreover, there is also a possibility that the tryptophan itself serves as a neuromodulator, and acts on the tryptophan receptor on the surrounding, that is, probably, on the pre-synapse side (new hypothesis, formed through experiments by the present inventors), to suppress many nervous system activities and consequently to inhibit motor-system output information in the intracerebral control circuit. In monitoring experiments of the tryptophan concentration in rat striatal extra-cellular fluid by using a micro-dialysis method, a high-concentration tryptophan release was observed during the fatigue, and this was allowed to quickly return to its basal level during the recovering period. In this manner, the tryptophan is allowed to effectively reflect the load and elapsed time of the fatigue (based upon Amino Acids, 17(1), p107, 1999; Neuroscience Res. Suppl. 23, S287, 1999, by the inventors et al. of the present application). In electrophysiological research using raphe nuclei neurons also, the neural firing was inhibited by the tryptophan (Federation Proc. 31:91-96, 1972), and the inventors, et al. of the present application have confirmed that rats into the brain of which the tryptophan (1 mM/30 min) has been continuously injected by using a micro-dialysis method will exhibit the fatigue in the central nervous system or muscles in very early stage (Amino Acids, 21(1), p55, 2001). Here, of course, it has been reported that the 5-HT itself suppresses the firing of cerebral cortex neurons (Brain Research, 231: 93-108, 1982). In this manner, in the fatigue in the muscles also, it is clear that the fatigue is greatly dependent on the fatigue in the central nervous system.

As described above, the fatigue in the central nervous system is dependent on the tryptophan concentration in the brain, and it is possible to suppress the fatigue in the central nervous system by inhibiting the transfer of the tryptophan into the brain. Here, the BCAA is allowed to function as an inhibitory substance against the L-system transporter on BBB, and the BCH is allowed to also function as a specific inhibitory substance against the L-system transporter on BBB; thus, the administration of the BCAA and BCH in a combined manner makes it possible to effectively suppress the fatigue in the central nervous system.

Industrial Applicability

The present invention makes it possible to specifically contribute to the fatigue-recovering and the fatigue-prevention in the central nervous system (brain fatigue), and also greatly contribute to alleviation and prevention of cerebrotonia fatigue caused by, for example, computer work and work in a space environment, which will be developed in the future.

Moreover, in accordance with rats for use in the fatigue model in the central nervous system, it is possible to confirm a function of the tryptophan with respect to brain fatigue (by the use of the tryptophan-deficient rats), and also to eliminate the influences of the tryptophan exerted in the brain due to endogenous albumin (by the use of analbuminemia rats). For this reason, by measuring the exercising capability of these rats by the use of a treadmill, it becomes possible to easily examine influences of various substances, such as a fatigue inhibitory substance relating to the central nervous system, to be exerted on the central nervous system.

1. An agent for recovering from fatigue of the central nervous system that is characterized by containing a branched-chain amino acid.

2. An agent for recovering from fatigue of the central nervous system that is characterized by containing a 2-aminobicyclo-[2,2,1]-heptane-2-carboxylic acid.

3. An agent for recovering from fatigue of the central nervous system that is characterized by containing a branched-chain amino acid and a 2-aminobicyclo-[2,2,1]-heptane-2-carboxylic acid.

4. A fatigue preventive agent for the central nervous system that is characterized by containing a branched-chain amino acid.

5. A fatigue preventive agent for the central nervous system that is characterized by containing a 2-aminobicyclo-[2,2,1]-heptane-2-carboxylic acid.

6. A fatigue preventive agent for the central nervous system that is characterized by containing a branched-chain amino acid and a 2-aminobicyclo-[2,2,1]-heptane-2-carboxylic acid.

7. A food for recovering from fatigue of the central nervous system that is characterized by containing a branched-chain amino acid.

8. A food for recovering from fatigue of the central nervous system that is characterized by containing a 2-aminobicyclo-[2,2,1]-heptane-2-carboxylic acid.

9. A food for recovering from fatigue of the central nervous system that is characterized by containing a branched-chain amino acid and a 2-aminobicyclo-[2,2,1]-heptane-2-carboxylic acid.

10. A food for preventing fatigue of the central nervous system that is characterized by containing a branched-chain amino acid.

11. A food for preventing fatigue in the central nervous system that is characterized by containing a 2-aminobicyclo-[2,2,1]-heptane-2-carboxylic acid.

12. A food for preventing fatigue in the central nervous system that is characterized by containing a branched-chain amino acid and a 2-aminobicyclo- [2,2, 1]-heptane-2-carboxylic acid.

13. An inhibitory substance screening method of fatigue in the central nervous system that is characterized by the step of:
measuring a fatigue-inhibiting degree by treadmill running tests using analbuminemia rats.

14. An inhibitory substance screening method of fatigue in the central nervous system that is characterized by the step of:
measuring fatigue-inhibiting degree by treadmill running tests using a tryptophan-deficient rat.

15. A fatigue-model rat for use as a fatigue model in the central nervous system, which is prepared as an analbuminemia rat.

16. A fatigue-model rat for use as a fatigue model in the central nervous system, which is prepared as a tryptophan-deficient rat.
